(19) United States
(12) Patent Application Publication
(10) Pub. No.: US 2026/0216214 A1
(43) Pub. Date: Jul. 30, 2026

(54) ANALGESIC COMPOSITIONS, METHOD OF MANUFACTURE AND USES THEREOF

(71) Applicant: University of Connecticut, Farmington, CT (US)

(72) Inventors: Lakshmi S. Nair, New Britain, CT (US); Devika Kishnan, New Britain, CT (US); Mu-Ping Nieh, Mansfield, CT (US); Joseph Walker, Avon, CT (US)

(21) Appl. No.: 19/139,597

(22) PCT Filed: Dec. 18, 2023
(86) PCT No.: PCT/US2023/084547

§ 371 Date: Jun. 16, 2025

Related U.S. Application Data
(60) Provisional application No. 63/433,044, filed on Dec. 16, 2022.

(51) Int. Cl. A61K 31/00; A61K 31/164; A61K 45/06; A61K 47/54

(52) U.S. Cl. CPC A61K 31/658; A61K 31/164; A61K 45/06; A61K 47/544

(57) ABSTRACT

Disclosed herein is an analgesic composition comprising a therapeutically effective amount of at least one endocannabinoid, at least one cannabinoid, or a combination of the endocannabinoid and cannabinoid and at least one local anesthetic; where the 2024/130236 endocannabinoid comprises one or more endocannabinoids, one or more analogues of the endocannabinoids, one or more pharmaceutically acceptable salts of the endocannabinoid, or a combination thereof; where the cannabinoid comprises one or more cannabinoids, one or more analogues of the cannabinoids, one or more pharmaceutically acceptable salts of the cannabinoid, or a combination thereof; where at least one of the endocannabinoid or the cannabinoid is in the form of particles.

### CROSS-REFERENCE TO RELATED APPLICATIONS

This disclosure claims priority to U.S. Provisional Application No. 63/433,044, filed on Dec. 16, 2022, the entire contents of which are incorporated herein in their entirety.

### GOVERNMENT SUPPORT

The invention was made with government support under grant numbers R01-AR075143 awarded by the National Institutes of Health. The government has certain rights in the invention.

### BACKGROUND

This disclosure relates to analgesic compositions, methods of manufacture thereof and to uses thereof. In particular, this disclosure relates to compounds, compositions, and methods for modulating pain, and their use in the prevention and/or treatment of pain and pain related illnesses.

Medications such as pain relievers and anti-inflammatory drugs may be prescribed to control pain and inflammation. Oral non-steroidal, anti-inflammatory drugs include medications classified as non-steroidal anti-inflammatory drugs (NSAIDs) such as ibuprofen, naproxen, and aspirin, which are commonly used to alleviate pain and reduce inflammation. However, these have serious side effects such as gastrointestinal problems. Opioids, while potent pain relievers, are a separate class of medications and are not classified as NSAIDs. However, these opioids can have serious side effects, such as the risk of addiction, respiratory depression, and constipation. Local anesthetics (LA) are potential molecules for managing pain; this is a non-addictive option, but these anesthetics are short acting. They also exhibit side effects such as local inflammation.

Osteoarthritis is a degenerative joint disorder characterized by a gradual breakdown of cartilage, the tissue that cushions the ends of bones within a joint. As the cartilage wears away, bones begin to rub against each other, leading to pain, stiffness, and reduced joint flexibility. This condition commonly affects weight-bearing joints such as the knees, hips, and spine, but it can also impact other joints over time. The primary risk factors for osteoarthritis include aging, genetics, joint injuries, and obesity. While there is no cure for osteoarthritis, various treatment options aim to manage symptoms and improve joint function. Non-pharmacological approaches include exercise, physical therapy, and weight management to alleviate stress on joints.

In severe cases, surgical interventions like joint replacement may be considered. Overall, a comprehensive approach involving lifestyle modifications, therapeutic interventions, and medical management is often employed to enhance the quality of life for individuals dealing with osteoarthritis.

There is a therefore a need for improved compositions that offer simplified routes for better pain management.

### SUMMARY

Disclosed herein is an analgesic composition comprising a therapeutically effective amount of at least one endocannabinoid, at least one cannabinoid, or a combination of the endocannabinoid and cannabinoid; where the endocannabinoid comprises one or more endocannabinoids, one or more analogues of the endocannabinoids, one or more pharmaceutically acceptable salts of the endocannabinoid, or a combination thereof; where the cannabinoid comprises one or more cannabinoids, one or more analogues of the cannabinoids, one or more pharmaceutically acceptable salts of the cannabinoid, or a combination thereof; where at least one of the endocannabinoid or the cannabinoid is in the form of particles.

Disclosed herein too is a method of reducing joint inflammation, swelling and/or pain in a subject, the method comprising administering a therapeutically effective amount of at least one endocannabinoid, at least one cannabinoid, or a combination of the endocannabinoid and cannabinoid to the subject; where at least one of the endocannabinoid or the cannabinoid is in the form of a nanoparticle and/or microparticle; and wherein the endocannabinoid comprises one or more endocannabinoids, one or more analogues of the endocannabinoids, one or more pharmaceutically acceptable salts of the endocannabinoid, or a combination thereof, where the cannabinoid comprises one or more cannabinoids, one or more analogues of the cannabinoids, one or more pharmaceutically acceptable salts of the cannabinoid, or a combination thereof.

### BRIEF DESCRIPTION OF THE FIGURES

FIG. 1 depicts the locations in the knee where the periarticular injections of only bupivacaine were administered;

FIG. 2 is a graph of paw withdrawal threshold versus time for Von Frey filaments in MIA induced OA rats treated with intraarticular injection of bupivacaine HCL (circles) and periarticular injection bupivacaine HCL at the four locations shown in FIG. 1 (squares); showing significantly prolonged pain relief with periarticular injection compared to intraarticular injection.

FIG. 3 is a graph that depicts the change from the baseline pre-injury values of the animals versus time in days. The figure shows that irrespective of the number of periarticular injections (1 vs 2 vs 4), all the periarticular injections provided prolonged pain relief compared to the disease control;

FIG. 4 is a transmission electron micrograph of the nanoparticles obtained from transmission electron microscopy;

FIG. 5 shows formation of PEA nanoparticles loaded with local anesthetic (bupivacaine HCl) at a mass ratio of DPPC: PEA: bupivacaine HCL=1:1:1;

FIG. 6 depicts the fold change for the gene expression of IL1β, TNF-α and IL6 and shows that the endocannabinoid dissolved in DMSO reduces the inflammatory response of macrophages;

FIG. 7 shows that PEA nanoparticles exhibit superior anti-inflammatory effect in reducing pro-inflammatory cytokine expression in IL1β and an increasing analgesic marker IL-10 when compared to PEA dissolved in DMSO at an equivalent concentration of 250 μM in LPS activated macrophages;

FIG. 8 is a graph depicting joint diameter on day 3 and day 7; showing that combining local anesthetic with PEA nanoparticles significantly reduced inflammation as characterized by joint diameter here;

FIG. 9 is a graph that shows joint diameter measurements made in Sprague Dawley rats as a measure of in-vivo anti-inflammatory effect as a change from baseline pre-injury values. This confirms the ability of the combined approach (local anesthetic (bupivacaine) and PEA nanoparticles) to significantly reduce inflammation and reduce the toxicity of bupivacaine as depicted here wherein we used a higher dose compared to FIG. 8;

FIG. 10 depicts electronic Von Frey measurements done with treatment groups using bupivacaine HCL periarticularly injected at 2 locations alone and periarticular injection of PEA nanoparticles with bupivacaine HCL at 2 locations. The combination approach showed similar pain relief compared to bupivacaine alone group initially followed by prolonged pain relief demonstrating the unique advantages of the combination approach; and

FIG. 11 depicts electronic Von Frey measurements done with treatment groups using high dose of bupivacaine HCL alone and periarticular PEA nanoparticles with high dose bupivacaine HCL; It shows the significantly enhanced hypoalgesic and analgesic effect of the combination approach and thereby demonstrates the enhanced therapeutic efficacy of the combination approach.

### DETAILED DESCRIPTION

### Definitions

The term “endocannabinoid” encompasses at least one endocannabinoid, an endocannabinoid analogue, a pharmaceutically acceptable salt of an endocannabinoid, or a combination thereof. Endocannabinoids are naturally occurring cannabinoids produced by the body. The endocannabinoids primarily target cannabinoid receptors are located on the cell surface. The term endocannabinoid encompasses one or more endocannabinoids, one or more analogues of the endocannabinoid, one or more pharmaceutically acceptable salts of the endocannabinoid, or a combination thereof.

The term “cannabinoid” encompasses a group of chemical compounds that interact with the endocannabinoid system in the human body. They can be classified into three main types a) endocannabinoids; b) phytocannabinoids; and c) synthetic cannabinoids. Cannabinoids exert their effects by interacting with cannabinoid receptors, primarily CB1 and CB2 receptors, which are part of the endocannabinoid system. CB1 receptors are mainly found in the central nervous system, while CB2 receptors are more prevalent in peripheral tissues, particularly immune cells. The term cannabinoid encompasses one or more cannabinoids, one or more analogues of the cannabinoid, one or more pharmaceutically acceptable salts of the cannabinoid, or a combination thereof.

The term “local anesthetic” includes a local anesthetic, an analogue of the local anesthetic, a pharmaceutically acceptable salt of the local anesthetic, or a combination thereof.

An analogue of a chemical compound is essentially a related compound that shares some structural or functional similarities with the original compound.

The term “periarticularly” refers to the area around a joint. It describes the tissues, structures, or conditions in the vicinity of a joint. In a medical context, it is used to discuss features or occurrences that impact or involve the areas surrounding joints, such as periarticular swelling or periarticular inflammation.

The term “intraarticularly” refers to something occurring within or relating to the interior of a joint. It denotes actions, conditions, or interventions that take place within the confines of a joint space. In medical contexts, this term might be used to describe procedures, medications, or issues that directly involve the interior of a joint, such as intraarticular injections of medication for pain relief or procedures aimed at addressing conditions within the joint capsule.

Inflammation is a complex biological response that occurs when the body's tissues are damaged. It is a protective mechanism involving the immune system, blood vessels, and various signaling molecules. The primary goals of inflammation are to remove the cause of cell injury, clear out damaged cells and tissues, and initiate tissue repair.

Swelling is one of the visible signs of inflammation. It refers to the enlargement or distension of a body part, typically due to the accumulation of fluid. Swelling occurs as a result of increased permeability of blood vessels, allowing fluid and immune cells to move from the bloodstream into the affected tissues.

A lipid molecule is a diverse group of organic compounds that are insoluble or poorly soluble in water but soluble in organic solvents. Lipids serve various functions in living organisms, including energy storage, insulation, and serving as structural components of cell membranes. Lipids are characterized by their hydrophobic (water-repelling) nature, which is due to their nonpolar hydrocarbon chains.

Nanoparticles as described herein include particles having a particle size of about 0.5 to about 100 nanometers. The diameter of the particles is generally considered to be a measure of particle size. Particle size may be determined by small angle xray scattering, scanning electron microscopy and/or transmission electron microscopy.

Microparticles as described herein include particles having a particle size of about 100.1 to about 100,000 nanometers. The diameter of the particles is generally considered to be a measure of particle size. Particle size may be determined by small angle xray scattering, scanning electron microscopy and/or transmission electron microscopy. The term “particles” as used herein encompasses both nanoparticles and microparticles. The term “particle” is used to describe phase separated endocannabinoids, cannabinoids and/or local anesthetics that are dispersed in a solution for administration into the body of a living being. The solution of the particles may be considered to be a suspension.

The term “therapeutically effective” is used to describe the efficacy of a pharmaceutical or therapeutic intervention. It refers to the ability of a particular composition or method to produce a beneficial or desired therapeutic effect in the treatment, prevention, or alleviation of a specific medical condition or disease.

“Hypoalgesic” is a term used in the context of pain perception and physiology. It refers to a reduction or suppression of pain sensitivity. Specifically, a hypoalgesic effect is characterized by a diminished response to painful stimuli, resulting in a lower perception of pain.

“Analgesic” is a term used to describe substances or interventions that alleviate or reduce pain. An analgesic is any agent or method that diminish or reduce pain sensation.

Von Frey data typically refers to experimental data collected using the von Frey filaments or hairs, which are instruments commonly used in neuroscience and behavioral research to assess mechanical sensitivity or tactile allodynia (an increased sensitivity to touch, often associated with pain or discomfort) in animals, particularly rodents. The von Frey test involves applying calibrated filaments of varying forces to the skin of an animal, usually the hind paw of a rodent. The animal's response is overserved to determine its sensitivity to mechanical stimuli. The data collected during von Frey testing may include a) Threshold Response: The force or bending pressure at which the animal first responds to the filament. This is often referred to as the “withdrawal threshold” and is an indicator of the animal's sensitivity to touch; b) Response Frequency: The number of positive responses (e.g., paw withdrawal, licking, or shaking) to the application of the von Frey filaments. This information provides a measure of the intensity or frequency of the animal's response to the mechanical stimulus; and c) Response Latency: The time it takes for the animal to respond after the application of the von Frey filament. A shorter response latency may indicate increased sensitivity.

Disclosed herein is an anti-inflammatory and analgesic composition (herein analgesic composition) that comprises a therapeutically effective amount of at least one endocannabinoid, at least one cannabinoid, or a combination of the endocannabinoid and cannabinoid, where at least one of the endocannabinoid or the cannabinoid is in the form of nano or micro-particles (hereinafter particles). In a preferred embodiment, the endocannabinoid is in the form of nanoparticles. In another preferred embodiment, the analgesic composition comprises an aqueous suspension of the particles. This invention describes the anti-inflammatory and analgesic effects of described compositions using osteoarthritis as a pain model.

Disclosed herein too is an analgesic composition that displays anti-inflammatory, analgesic and anti-swelling properties that comprises a therapeutically effective amount of a local anesthetic, and a therapeutically effective amount of at least one endocannabinoid, cannabinoid, or a combination thereof; where at least one of the endocannabinoid, the cannabinoid, the local anesthetic or any combination of the endocannabinoid, the cannabinoid and the local anesthetic are in the form of nanoparticles and/or microparticles (together encompassed by the term “particles”). In a preferred embodiment, at least two of the endocannabinoids, the cannabinoids and the local anesthetic are in the form of nanoparticles and/or microparticles. In another preferred embodiment, the endocannabinoid and the local anesthetic coexist in a single nanoparticle and/or microparticle. In a preferred embodiment, the analgesic composition comprises an aqueous suspension of the nanoparticles and/or microparticles.

As previously noted, the term “endocannabinoid” encompasses one or more endocannabinoids, one or more analogues of the endocannabinoid, one or more pharmaceutically acceptable salts of the endocannabinoid, or a combination thereof. As previously noted too, the term “cannabinoid” encompasses one or more cannabinoids, one or more analogues of the cannabinoid, one or more pharmaceutically acceptable salts of the cannabinoid, or a combination thereof. As previously noted too, the term “local anesthetic” includes one or more local anesthetic, one or more analogues of the local anesthetic, one or more pharmaceutically acceptable salts of the local anesthetic, or a combination thereof.

Some endocannabinoids (e.g., palmitoylethanolamide) display anti-inflammatory properties but are very hydrophobic and hence have low bioavailability when taken orally. The most common method to increase bioavailability is by micronizing these endocannabinoid particles for oral uptake. However, the quantities available for oral uptake are often not enough to restore local levels at the site of discomfort. A local delivery of the endocannabinoid would be beneficial in restoring it to natural levels at the site of discomfort.

Endocannabinoids have been encapsulated in carriers and used as anti-inflammatories at low loadings (less than about 10 wt %). However, these efforts have not been entirely successful because of the concentration dependent nature the anti-inflammatory relief. To overcome these problems, the endocannabinoids and/or cannabinoids have been incorporated into the analgesic composition in loadings of about 0.1 weight percent (wt %) to about 90 wt %, based on a total weight of the analgesic composition. The endocannabinoids and/or cannabinoids are present in the form of nano and/or micro-particles. The analgesic composition may comprise one or more lipids in addition to the endocannabinoids and/or cannabinoids. The lipids facilitate the formation of endocannabinoid and/or cannabinoid particles (e.g., the nanoparticles and the microparticles).

In an embodiment, the analgesic composition is advantageous in that (with or without the local anesthetic) it may be administered periarticularly in a single effective dose at a single location and provides superior or equivalent analgesic relief as compared with the periarticular or intraarticular administering of multiple doses in multiple locations around or in a joint respectively. In an embodiment, a single dose of the analgesic composition (with or without the local anesthetic) administered periarticularly displays the same analgesic relief efficacy as two or more doses administered periarticularly or intraarticularly administered around or in a joint. In yet another embodiment, a single dose of the analgesic composition (with or without the local anesthetic) administered periarticularly displays the same analgesic relief efficacy as four or more doses administered periarticularly or intraarticularly administered around or in a joint.

Disclosed herein too is a method to reduce joint inflammation, swelling and/or pain in a subject, the method comprising providing and administering a therapeutically effective amount of at least one endocannabinoid, at least one cannabinoid, or a combination of the endocannabinoid and cannabinoid to the subject; where at least one of the endocannabinoid or the cannabinoid is in the form of a particle. The composition comprises particles of the endocannabinoid and/or local anesthesia in an aqueous suspension. The composition may be administered periarticularly in a single or multiple doses as detailed above.

Disclosed herein too is a method to reduce joint inflammation, swelling and/or pain in a subject, the method comprising providing and administering a therapeutically effective amount of a local anesthetic, and at least one endocannabinoid, at least one cannabinoid, or a combination of the endocannabinoid and cannabinoid to the subject; where at least one of the endocannabinoid, the cannabinoid, the local anesthetic, or any combination of the endocannabinoid, the cannabinoid, or the local anesthetic is in the form of a particle e.g., a nanoparticle and/or a microparticle). In an embodiment, at least two of the endocannabinoid, the cannabinoid and the local anesthetic are in the form of particles. In a preferred embodiment, the endocannabinoid and the local anesthetic coexist in a single particle. In a preferred embodiment, the analgesic composition comprises an aqueous suspension of the particles.

The disclosed method presents a minimally invasive approach to reduce pain. For e.g., osteoarthritic (OA) pain via periarticular injections. This will be more cost effective, have higher patient compliance and allow patients to continue physical therapy for permanent improvement of joint function. The approach helps to assist patients to get a minimally invasive treatment for osteoarthritic (OA) pain management. This may be particularly of interest to patients who undergo a total knee replacement. It may also be of interest to patients experiencing periarticular pain. This approach improves pain management strategies and helps prepare patients for physical therapy after surgery or after damage to a joint.

The analgesic composition comprises the endocannabinoid, an optional cannabinoid, an optional local anesthetic and a liquid. As noted above, the endocannabinoid, the cannabinoid (if present), the local anesthetic (if present) are dispersed as particles in the liquid. The analgesic composition may therefore be an emulsion. In an embodiment, the liquid is an aqueous solution that is biocompatible (i.e., non-toxic) with the body of a living being. It is to be noted that the analgesic composition is never in solution form. In other words, the endocannabinoid is always in particle form in the analgesic composition. In some embodiments, the cannabinoid and the local anesthetic are also in particle form in the analgesic composition. In some embodiments, the cannabinoid and the local anesthetic are not in particle form in the analgesic composition, while the endocannabinoid is in particle form. The cannabinoid and the local anesthetic may be dissolved in the liquid while the endocannabinoid is in particle form.

In an embodiment, the analgesic composition may contain lipid molecules in addition to the endocannabinoid, the cannabinoid, the local anesthetic and the liquid. The analgesic composition may also contain analogues of the endocannabinoid, the cannabinoid, and/or the local anesthetic in addition to endocannabinoid, the cannabinoid and/or the local anesthetic.

Endocannabinoids are naturally occurring compounds within the bodies of living beings that interact with the endocannabinoid system (ECS). The ECS is a complex cell-signaling system that plays a crucial role in regulating a range of physiological processes to maintain homeostasis, or balance, in the body. Endocannabinoid ligands include endogenously produced lipids that activate two distinct “direct” endocannabinoid receptors, CB1 and CB2. CB1 receptors are primarily found in the central nervous system, including the brain. The activation of CB1 receptors can modulate the perception of pain and affect the transmission of pain signals.

CB2 receptors are mainly located in peripheral tissues, particularly in immune cells. CB2 receptor activation is associated with anti-inflammatory effects, which can contribute to pain relief in conditions involving inflammation.

Endocannabinoids and endocannabinoid like molecules that interact with the ECS system to provide anti-inflammatory, analgesic (pain-relieving) and/or swelling relief include N-acylethanolamines (NAEs), and N-acyl amino acids their analogues. N-acylethanolamines are fatty acid amides that are naturally produced in the body in response to various stimuli, including inflammation. Examples of N-acylethanolamines and its analogues that can be used for their anti-inflammatory effect include anandamide (AEA) 2-arachidonoylglycerol (2-AG), 2-oleoyl glycerol (2-OG), palmitoylethanolamide (PEA), oleoylethanolamide (OEA), linoleoylethanolamide (LEA), adelmidrol (N,N′-Bis(2-hydroxyethyl) nonanediamide), N-stearoyl ethanolamine (SEA), N-arachidonoyl glycine (NArGly), N-arachidonoylserine (NArS), N-acyl taurine, N-acyl dopamine, N-acyl alanine, or a combination thereof.

In an exemplary embodiment, the preferred endocannabinoid used in the analgesic composition is N-palmitoylethanolamide. A main target of N-palmitoylethanolamide is the peroxisome proliferator-activated receptor alpha (PPAR-α). N-palmitoylethanolamide also has affinity to cannabinoid-like G-coupled receptors GPR55 and GPR119. Palmitoylethanolamide (or other structurally related N-acylethanolamines) enhances anandamide activity by an “entourage effect”.

Cannabinoids are chemical compounds found in the cannabis plant. Some cannabinoids may also be used for their anti-inflammatory effect. Examples of such cannabinoids or cannabinoid analogues include cannabigerol (CBG), cannabichromene (CBC), trans-beta caryophyllene (β-caryophyllene), curcumin, honokiol, cannabinol, cannabidiol, delta 9-tetrahydrocannabinol, delta 8-tetrahydrocannabinol, hydroxy-tetrahydrocannabinol, 11-hydroxy-9-tetrahydrocannabinol, levonantradol, delta 11-tetrahydrocannabinol, tetrahydrocannabivarin, dronabinol, nabilone, ajulemic acid, triaryl bis-sulfone, luteolin, or a combination thereof.

In an embodiment, the endocannabinoids and/or the cannabinoids used in the analgesic compositions may be in the form of particles (e.g., nanoparticles and/or microparticles). In an embodiment, at least one of the endocannabinoids or the cannabinoids used in the analgesic compositions may be in the form of particles. Nanoparticles are small particles with dimensions in the nanometer range. Microparticles have particle sizes of about 100.1 to about 100,000 nanometers. These types of particles are defined further below. These particles (nanoparticles and microparticles) may have different geometries such as, for example, spherical, platelet-like, ellipsoidal, rod-like (e.g., elongated or cylindrical shape), cuboidal, triangular shape, core and shell, star-shaped particles (e.g., the particles have multiple arms radiating from a central core, or the like. In an embodiment, the particles have platelet-like shapes (i.e., they are disc shaped).

The particle size distribution of particles may be unimodal or multimodal. Multimodal particle size distributions may include binodal, trinodal or multinodal distributions. Multinodal may include 4 or more distributions in particle size. The particles have been found to be more efficacious in providing anti-inflammatory relief to activated macrophages than a corresponding solution (where the endocannabinoids are completely dissolved in the liquid) that contains the same amount of the endocannabinoid (with or without a local anesthetic).

As noted above, the analgesic composition can contain particles. These particles include nanoparticles, microparticles, or simultaneously contain both nanoparticles and microparticles. In other words, the analgesic composition can contain particles that have particle sizes that extend from the nanoparticle range to the microparticle range. In an embodiment, the nanoparticles have a particle size of less than about 100 nanometers, preferably about 2 to about 80 nanometers and more preferably about 5 to about 50 nanometers. The microparticles have a size of about 100.1 to about 100,000 nanometers, preferably about 200 to about 800 nanometers, and more preferably about 250 to about 400 nanometers.

In an embodiment, the analgesic composition may contain endocannabinoid and/or cannabinoid particles suspended in an aqueous solution. The endocannabinoid particles may be present in the analgesic composition in an amount of about 0.1 to about 97 weight percent (wt %), about 0.2 to about 85 wt %, about 1 to about 80 wt %, about 2 to about 70 wt %, about 3 to about 60 wt %, about 3.5 to about 55 wt %, about 4 to about 50 wt %, about 4.5 to about 45 wt %, about 5 to about 40 wt %, about 5.5 to about 35 wt %, about 6 to about 30 wt %, about 6.5 to about 25 wt %, about 7 to about 20 wt %, about 7.5 to about 18 wt %, about 8 to about 15 wt %, and about 9 to about 14 wt %, based on a total weight of the analgesic composition. The cannabinoid particles may be present in the analgesic composition in an amount of 0 to about 97 weight percent (wt %), 0.1 to about 95 weight percent (wt %), about 0.2 to about 85 wt %, about 1 to about 80 wt %, about 2 to about 70 wt %, about 3 to about 60 wt %, about 3.5 to about 55 wt %, about 4 to about 50 wt %, about 4.5 to about 45 wt %, about 5 to about 40 wt %, about 5.5 to about 35 wt %, about 6 to about 30 wt %, about 6.5 to about 25 wt %, about 7 to about 20 wt %, about 7.5 to about 18 wt %, about 8 to about 15 wt %, and about 9 to about 14 wt %, based on a total weight of the analgesic

The analgesic composition may optionally contain a local anesthetic. The local anesthetic facilitates a reduction in pain. The local anesthetics may induce a reversible loss of sensation in a specific area of the body. They work by blocking nerve signals in the vicinity of the application, leading to temporary numbness or loss of pain sensation. In an embodiment, the local anesthetic can act synergistically with the endocannabinoid and/or the cannabinoid to provide more efficient and safer therapy. In an embodiment, the endocannabinoid and/or the cannabinoid can serve as a carrier for the sustained delivery of a local anesthetic. The endocannabinoid and/or the cannabinoid can reduce the toxicity of the local anesthetic when the analgesic composition is administered in vivo periarticularly and/or intraarticularly to a joint.

Examples of local anesthetics include lidocaine (xylocaine), bupivacaine (marcaine), mepivacaine (carbocaine), ropivacaine (naropin), procaine (novocain), articaine (septocaine), chloroprocaine, Qx-314, or the like, or a combination thereof. A preferred local anesthetic is bupivacaine. In an embodiment, a preferred local anesthetic for use in a joint such as the knee is bupivacaine. In an embodiment, the local anesthetic may be entirely soluble in the liquid. In another embodiment, the local anesthetic may be present as particles (nano or microparticles in the analgesic composition.

In an embodiment, the analgesic composition may contain the local anesthetic in an amount of 0 to about 60 weight percent (wt %), about 0.005 to about 50 wt %, about 0.1 to about 45 wt %, about 1 to 47 wt %, about 2 to about 40 wt %, about 3 to about 35 wt %, based on a total weight of the analgesic composition.

The analgesic composition may also contain endocannabinoid lipids alone or with supporting lipid or lipids such as phospholipids. An example of a lipid that may be used in the analgesic composition includes lipid molecule 1,2-dipalmitoyl-sn-glycero-3-phosphocholine [DPPC], 1,2-dimyristoyl-sn-glycero-3-phosphocholine [DMPC], 1,2-distearoyl-sn-glycero-3-phosphocholine [DSPC], 1,2-dioleoyl-sn-glycero-3-phosphocholine [DOPC], or a combination thereof.

The analgesic composition may contain the lipid in an amount of about 0.01 to about 95 weight percent (wt %), preferably about 30 to about 60 wt %, based on a total weight of the analgesic composition. For example, the analgesic composition may contain the lipid in an amount of about 1 to about 95 wt %, about 5 to about 90 wt %, about 10 to about 80 wt %, about 15 to about 75 wt %, about 20 to about 70 wt %, about 25 to about 70 wt %, about 30 to about 65 wt %, about 35 to about 60 wt %, about 40 to about 55 wt %, about 45 to about 50 wt %, about 20 to about 60 wt %, and about 25 to about 50 wt %, based on a total weight of the analgesic composition.

In an embodiment, when an endocannabinoid, a lipid and a local anesthetic are used in an analgesic composition, each of the endocannabinoid, the lipid and the local anesthetic may each be used in an amount of about 20 to about 40 wt %, preferably about 30 to about 35 wt %, based on a total weight of the endocannabinoid, the lipid and the local anesthetic.

In an embodiment, in a suspension, the endocannabinoid and/or the cannabinoid and/or the local anesthetic can be present in an amount of about 0.1 to about 25 volume percent, based on a total volume of the entire suspension.

The liquid present in the analgesic composition is preferably one that is biocompatible and does not solvate the endocannabinoid. Water or an aqueous solution is a preferred liquid for the endocannabinoid particles. Some endocannabinoids are known to be crystalline. Endocannabinoids are also water-insoluble and would have to be solvated in an organic solvent (that may not be compatible with the surrounding tissue into which it is injected). It is therefore desirable and efficacious to convert the endocannabinoids into particles in an aqueous liquid. Suitable liquids are water, phosphate-buffered saline (PBS) or saline. The saline solution preferably has a concentration of 0.9% weight per unit volume (w/v) sodium chloride (i.e., it is considered isotonic, meaning it has a similar osmotic pressure to bodily fluids). The saline solution may be hypertonic (higher concentration of sodium chloride than 0.9%) or hypotonic (lower concentration of sodium chloride than 0.9%) if desired.

Other organic solvents that are not toxic may be mixed with the liquid in small quantities if desired. Examples of such organic solvents include ethanol, polyethylene glycol, polypropylene glycol, or a combination thereof. These organic solvents may be used to alter the size of the particles depending upon the application.

The liquid is present in the analgesic composition in an amount of about 0.5 to about 99.9 wt %, about 1 to about 95 wt %, about 5 to about 90 wt %, about 10 to about 80 wt %, about 15 to about 75 wt %, about 20 to about 70 wt %, about 25 to about 70 wt %, about 30 to about 65 wt %, about 35 to about 60 wt %, about 40 to about 55 wt %, about 45 to about 50 wt %, about 20 to about 60 wt %, and about 25 to about 50 wt %, based on a total weight of the analgesic composition.

In an embodiment, the endocannabinoid and/or the local anesthetic may each be used in an amount of about 0.01 to about 2 wt %, about 0.1 to about 1 wt %, and about 0.15 to about 1.5 wt %, based on a total weight of the analgesic composition, while the liquid may be used in an amount of about 95 to 99.9 wt %, about 96 to 99.8 wt %, based on a total weight of the analgesic composition.

### Manufacturing of the Particles

As noted above, the endocannabinoid and/or the cannabinoid may be present in the analgesic composition in the form of particles (e.g., nanoparticles or microparticles).

The endocannabinoid and/or the cannabinoid alone or with a supporting lipid or lipids such as phospholipids are mixed and dispersed in a first organic solvent. In an exemplary embodiment, the first organic solvent is an alcohol. The first organic solvent is then evaporated in vacuum overnight leaving behind a powder or film.

While the first solvent is listed above as being an alcohol, other solvents such as, for example ketones, dimethylsulfoxide, dimethylformamide, tetrahydrofuran, acetone, methyl ethyl ketone, ethylene glycol, and the like, or a combination thereof may be used.

Examples of alcohol include methanol, ethanol, propanol, butanol, or a combination thereof. It is desirable to remove all traces of solvents prior to the homogenization process that is described below.

The endocannabinoid and/or the cannabinoid and any supporting lipid is dissolved in a first solvent. The first solvent may be an alcohol such as methanol. The dissolution temperature is about 50 to about 80° C., preferably about 60 to about 75° C. The first solvent is evaporated under a vacuum.

The sample after the removal of methanol is mixed with sterile filtered water. For crystalline lipids, hot homogenization is performed. The hot homogenization process is conducted at a temperature of above the melting point of the lipid. For example, for PEA, a temperature of about 105 to about 110° C. was used under rotary agitation of about 5,000 to about 15,000 revolutions per minute (rpm), preferably about 8000 to about 10,000 rpm for a period of time of about 2 to about 30 minutes, preferably about 4 to about 15 minutes to form particles. In an embodiment, the particles in the water may constitute the analgesic composition. Alternatively, a local anesthetic may be added to the particle solution to form the analgesic solution. The local anesthetic may dissolve in the water (i.e., be soluble in the analgesic solution) or alternatively, be present in the form of particles.

Administration of analgesic composition in or around a joint The analgesic composition may be administered (i.e., injected) directly into a subject (a living being) at the site of pain, for instance, in a therapeutically effective amount periarticularly and/or intraarticularly around a damaged joint. In a preferred embodiment, the analgesic composition is administered in a therapeutically effective amount periarticularly to reduce inflammation, pain and/or swelling. In another embodiment, the analgesic composition without the local anesthetic may be administered (locally) in a therapeutically effective amount (dose) periarticularly and/or intraarticularly followed by a second dose that contains the local anesthetic also administered periarticularly and/or intraarticularly. In other words, the analgesic composition and the local anesthetic may be administered simultaneously (as a single therapeutically effective amount) or sequentially in two separate doses-the first dose being the analgesic composition and the second dose being the local anesthetic or vice versa.

In an embodiment, the analgesic composition may be delivered in a single dose in a therapeutically effective amount periarticularly. The single dose has been found to be as efficacious as two or more doses delivered periarticularly, preferably as efficacious as four or more doses delivered periarticularly.

In an embodiment, the analgesic composition periarticularly provides a prolonged hypoalgesic/analgesic effect for the local anesthetic. For example, a subcutaneous injection into 2 of the periarticular locations around the knee, namely the superior lateral and inferior medial, can provide similar duration of analgesic efficacy as the 4 locations injections. The injections were 50 μL each and the bupivacaine HCL (1.5 mg/ml) was dissolved in 0.9% saline. This experiment also indicated that only half the amount of bupivacaine HCL was enough to produce same analgesic efficacy with periarticular injection in 2 locations versus 4 locations.

The unique synergistic effects of the endocannabinoid and/or cannabinoids in conjunction with a local anesthetic enhances the analgesic efficacy and reduces the side effects of the local anesthetic. This facilitates safer and more efficient local anesthetic formulations. The formulation can replace current treatments where a local anesthetic is used, in order to decrease its inflammatory effect and prolong its anesthetic-hypoalgesic-analgesic effect. For instance, that analgesic composition disclosed herein may be used for pain relief for the following:
a. General surgery: colon, stomach, hernia, kidney, weight loss
b. Orthopedic surgery/Sports medicine: knee, hip, spine, foot, ankle, hand, shoulder
c. Oral surgery/dental procedures: wisdom teeth, jaw
d. OB-GYN/Breast surgery: hysterectomy, fibroid, breast, C-section (Caesarean section)
e. Anesthesia/Pain medicine: nerve blocks, epidurals

The analgesic composition is advantageous in that it can be administered in the periarticular regions around the knee to significantly prolong the analgesic efficacy of the local anesthetic. This indicates a synergy between the local anesthetic and the endocannabinoid/cannabinoid particles. The endocannabinoid/cannabinoid particles display higher anti-inflammatory properties than solutions of the endocannabinoid/cannabinoid (where the endocannabinoid/cannabinoid are completely dissolved in the liquid).

The analgesic compositions disclosed herein and the method of manufacture thereof are exemplified by the following non-limited examples.

### EXAMPLE

### Example 1

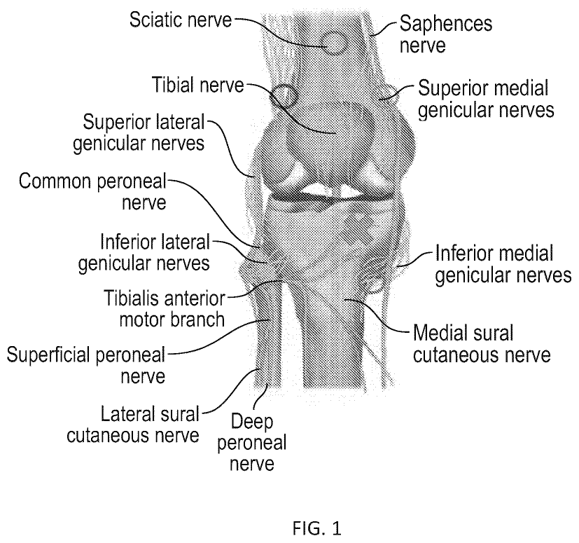

This example is conducted to demonstrate the effect of periarticular injections of only bupivacaine (1.5mg/mL) in four defined locations in the knee. The injections were 50 μL each and the Bupivacaine HCL (1.5 mg/mL) was dissolved in 0.9% saline. Each dose of 50 μL was administered at one of four locations-the superior medial, superior lateral, inferior medial and medial retinacular locations in the knee. FIG. 1 depicts the locations in the knee where the periarticular injections of only bupivacaine were administered.

Periarticular injections of local anesthetics into these specific locations can prolong the duration of analgesia compared to intra-articular injections into the knee joint. For example, the injection of bupivacaine (a local anesthetic) into the 4 locations defined above increases its duration of efficacy to 17 days compared with 3 days (for a MIA only group) with intraarticular injection of same amount of bupivacaine HCL in a rodent monoiodoacetate (MIA) induced osteoarthritis (OA) model. Monoiodoacetate is a chemical compound that is used to induce osteoarthritis-like symptoms in animal models. It is employed to study the mechanisms and potential treatments for osteoarthritis, a degenerative joint disease characterized by the breakdown of joint cartilage and underlying bone.

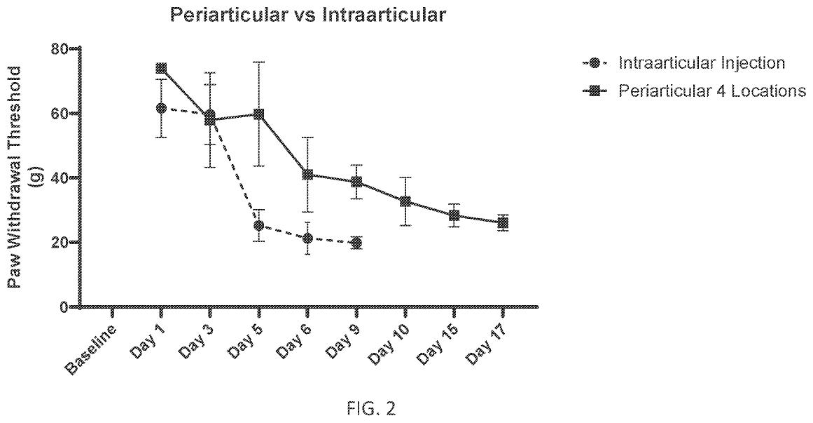

FIG. 2 is a graph of paw withdrawal threshold versus time for Von Frey filaments in MIA induced osteoarthritis (OA) rats treated with intraarticular injection of bupivacaine HCL (circles) and periarticular injection bupivacaine HCL at the four locations shown in FIG. 1 (squares). The graph shows significantly prolonged pain relief with the periarticular injection compared to intraarticular injection.

### Example 2

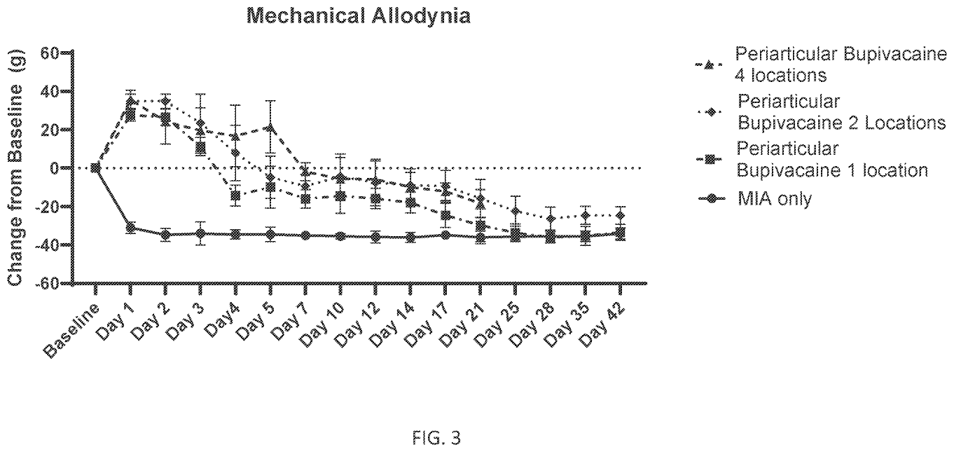

This example is a continuation of Example 1 and shows that periarticular injections into 2 of the periarticular locations around the knee, namely superior lateral and inferior medial, can provide similar duration of analgesic efficacy as the 4 locations injections (see FIG. 3). Periarticular injection at one location (superior lateral) showed similar analgesic effect as 2 and or 4 locations. FIG. 3 is a graph that depicts the change from the baseline pre-injury values of the animals versus time in days. The injections were 50 μL each of bupivacaine HCL. The bupivacaine HCL (1.5 mg/ml) is dissolved in 0.9% (w/v) saline. This example also indicates that only half the amount of bupivacaine HCL is enough to produce same analgesic efficacy with periarticular injection in 2 locations versus 4 locations. FIG. 3 depicts the paw withdrawal threshold as measured using Von Frey filaments. in MIA induced OA rats. The figure shows that irrespective of the number of periarticular injections (1 vs 2 vs 4), all the periarticular injections provided prolonged pain relief compared to the disease control.

### Example 3

This example was conducted to demonstrate how the nanoparticles were manufactured. PEA and another lipid molecule 1,2-dipalmitoyl-sn-glycero-3-phosphocholine [DPPC] (mass ratio of 1:1) were dissolved in methanol. The mixture was vortexed and if needed heated up to 60° C. to completely dissolve and disperse the PEA and 1,2-dipalmitoyl-sn-glycero-3-phosphocholine.

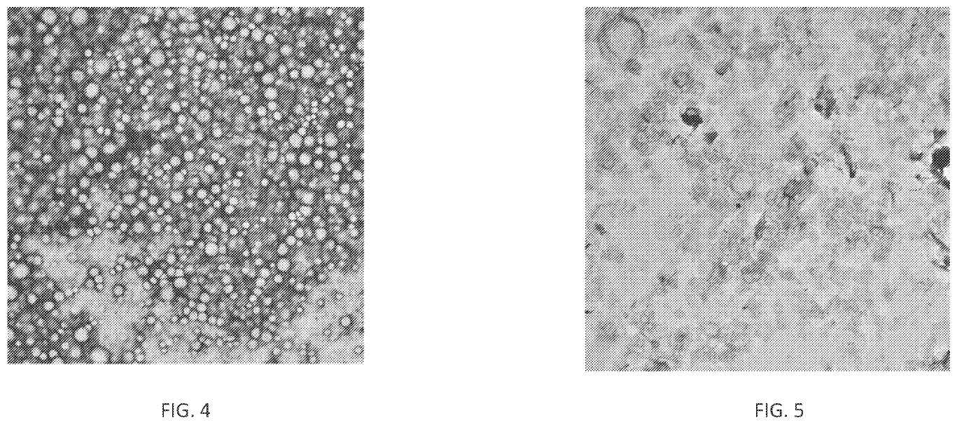

The sample was placed in a vacuum oven for overnight removal of organic solvent. The sample (lipids) was subjected to homogenization at high temperature, preferably 105-110° C. Sterile filtered water was added to the sample and the sample homogenized by mixing at 10,000 rpm for 3-10 minutes to form the nanoparticles. FIG. 4 is a transmission electron micrograph of the nanoparticles obtained from transmission electron microscopy [TEM]. The nanoparticles appear to have spherical vesicular structures.

### Example 4

This example was conducted to demonstrate the manufacturing of an analgesic composition that contained both the endocannabinoid particles and the local anesthetic. FIG. 5 shows formation of PEA nanoparticles loaded with local anesthetic (bupivacaine HCl) at a mass ratio of DPPC: PEA: bupivacaine HCL =1:1:1. The PEA bupivacaine NPs appear to have disc like structures.

### Example 5

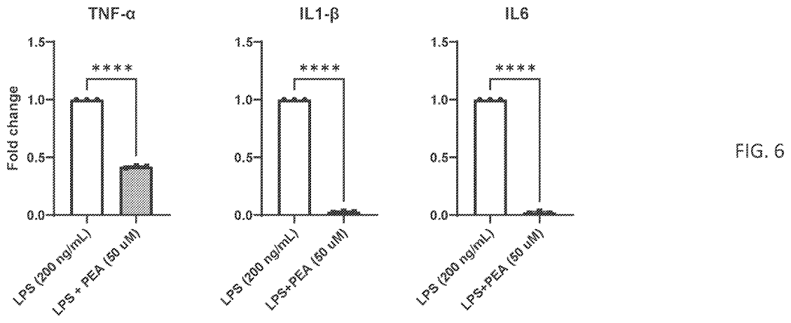

This example was conducted to demonstrate the anti-inflammatory and analgesic effects of the nanoparticles. Bone marrow macrophages were cultured for 24 hours, followed by treatment with lipopolysaccharides (LPS) from Escherichia coli O111:B4 (to activate macrophages) and the endocannabinoid molecule, dissolved in dimethyl sulfoxide (DMSO). After 24 hours of treatment, the cells are lysed, and the RNA collected. Gene expression is determined using RT-PCR. The expression of inflammatory cytokines such as Tumor Necrosis Factor Alpha (TNF-α), Interleukin-1-Beta (ILβ) and Interleukin-6 are used to determine the anti-inflammatory effect of the endocannabinoid solution. For example, macrophages were cultured for 24 hours and treated with LPS (200 ng/ml) and PEA (50 μM). After 24 hours, cells were lysed, and the RNA collected. Gene expression of ILβ, TNF-α and IL6 were significantly less than in the LPS alone control group demonstrating the anti-inflammatory effect of PEA. FIG. 6 depicts the fold change for the gene expression of ILβ, TNF-α−and IL6 and shows that the endocannabinoid dissolved in DMSO reduces the anti-inflammatory response of inflammatory cytokines. The ‘****’ indicates that p<0.0001 in an unpaired two tailed t-test with N=3 (three experimental repeats with three technical repeats each).

### Example 6

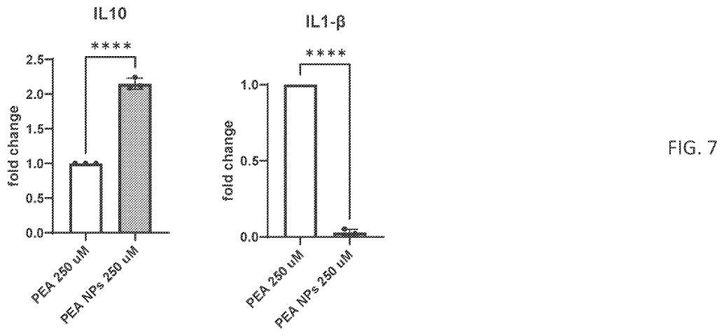

This example was also conducted to demonstrate the anti-inflammatory effect. Bone marrow macrophages are cultured for 24 hours, followed by treatment with lipopolysaccharides (to activate macrophages) and the endocannabinoid dissolved in DMSO or endocannabinoid nanoparticle formulation at the same concentration. 24 hours after treatment, the cells were lysed, and the RNA collected. Gene expression is determined using RT-PCR. The expression of inflammatory cytokines such as Tumor Necrosis Factor Alpha (TNF-α), and Interleukin-1-Beta (IL1β) is used to determine the anti-inflammatory effect of the endocannabinoid solution. For example, LPS activated macrophages (200 ng/ml) when treated with PEA nanoparticles (250 μM) showed significantly enhanced anti-inflammatory effect compared the PEA solution (250 μM, dissolved in DMSO) . FIG. 7 shows that PEA nanoparticles exhibit superior anti-inflammatory effect in reducing pro-inflammatory cytokine expression in IL1β and an increasing analgesic marker IL-10 when compared to PEA alone at an equivalent concentration of 250 μM in LPS activated macrophages.

### Example 7

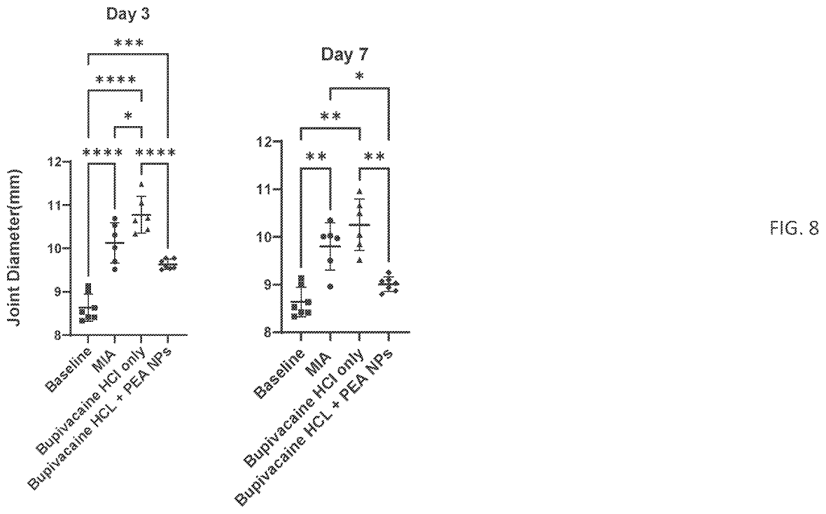

This example was conducted to determine in-vivo inflammation. An OA rodent model treated with endocannabinoid formulation and local anesthetic shows synergistic effect with reduction in inflammation near the joint. The joint inflammation is assessed via vernier caliper measurements. For example, female Sprague Dawley Rats were injected with 30 μL of mono-iodoacetate solution to induce OA. After 24 hours, the rats were periarticularly injected at 4 locations (50 μL/location): medial superior region and medial retinacular region received PEA NPs in 0.9% saline; lateral superior and medial inferior region received PEA nanoparticles+bupivacaine HCl (1.5 mg/ml) in 0.9% saline. The injections were done under X-ray guidance using a C-arm for increased precision. Inflammation was assessed using a vernier caliper at the knee joint. The data indicates that the PEA nanoparticles treated group saw significantly reduced inflammation at the knee joint compared to the periarticular bupivacaine alone group at day 3 and day 7. At day 3, periarticular bupivacaine injection significantly increased joint diameter or inflammation compared to the injection of MIA alone, showing increased inflammation due to bupivacaine injection. Both on days 3 and 7, periarticular injection of PEA nanoparticles along with bupivacaine showed significantly reduced joint diameter or joint inflammation compared to periarticular bupivacaine injected group showing the unique advantages of the combined therapy in addressing the issue of joint inflammation. At day 7, the PEA nanoparticles with bupivacaine group showed no significant differences in the joint diameter measurements (a measure of joint inflammation) compared to baseline (uninjured knee) (see FIG. 8) further demonstrating the unique advantage of the combined approach compared to bupivacaine alone. FIG. 8 shows joint diameter measurements in female Sprague Dawley rats post MIA injection followed by treatment with bupivacaine HCl only or using bupivacaine HCL in conjunction with PEA nanoparticles. Vernier caliper measurements show reduced inflammation in the bupivacaine+PEA nanoparticles group compared to the bupivacaine HCL only group at day 3 and day 7. At day 7, the inflammation in the bupivacaine+PEA nanoparticles group is not different from the baseline. At day 3, the inflammation in the bupivacaine HCl only group is higher than the MIA group. Statistical tests were conducted using only one way ANOVA with Brown Forsythe and Welsh tests. In the FIG. 8, * indicates p<0.05, while *** indicates p<0.001 and indicates that p<0.0001.

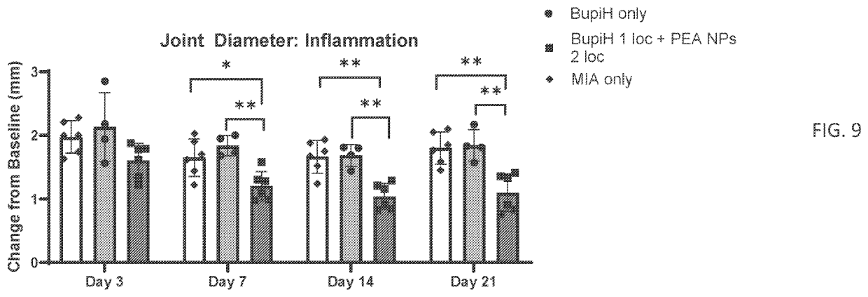

Similar to this finding, even in 3 mg/ml local anesthetic injection coadministration of local anesthetic with PEA-NP significantly reduces the knee inflammation. FIG. 9 is a graph depicting joint diameter upto 21 days. It shows that the coadministration of local anesthetic with PEA-NP significantly reduces the knee inflammation. FIG. 9 shows joint diameter measurements in female Sprague Dawley as a measure of in-vivo anti-inflammatory effect show as change from baseline (pre-injury values). The treatment groups are high does of bupivacaine HCl at 1 location (N=4), periarticular PEA nanoparticles at 2 locations +high does bupivacaine HCL at 1 location (N=5) with control of MIA (3 mg; N=6). The treatment groups with PEA nanoparticles show anti-inflammatory effects for 3 weeks. Statistical tests were conducted using only one way ANOVA with Brown Forsythe and Welsh tests. In the FIG. 9, * indicates p<0.05, while ** indicates p<0.01 and ** indicates that p<0.001.

When however, the bupivacaine concentration was increased (for instance from 0.15% to 0.3%), there was a significant reduction in the hypoalgesic and analgesic effect of bupivacaine. The presence of PEA nanoparticles completely rescued the effect, by removing the toxicity of higher bupivacaine concentration. This confirms the ability of the combination approach (local anesthetic (bupivacaine) and PEA nanoparticles) to significantly reduce inflammation and reduce the toxicity of bupivacaine as depicted here wherein we used a higher dose compared to FIG. 8.

### Example 8

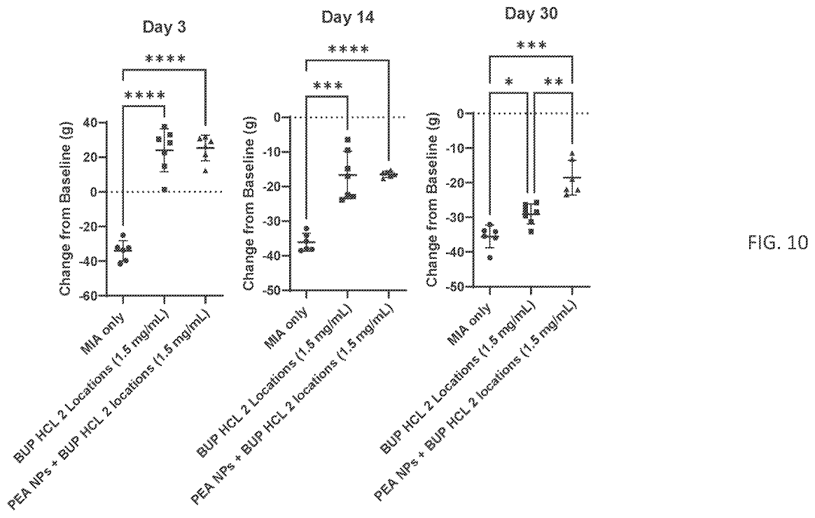

This example was conducted to determine in-vivo pain response. An OA rodent model treated with endocannabinoid formulation and local anesthetic shows synergistic effect with prolonged or similar pain relief depending on the concentration as compared to local anesthetic alone. Pain response was assessed using Von Frey filaments that measure mechanical allodynia. Paw withdrawal threshold was measured using Electronic Von Frey [IITC]. For example, female Sprague Dawley Rats were injected with 30 μL of mono-iodoacetate solution to induce OA. After 24 hours, the rats were periarticularly injected at 2 locations (50 μL/location): medial superior region and medial retinacular region received PEA nanoparticles+bupivacaine HCl (1.5 mg/ml) in 0.9% saline. The results were compared to rodents with 2 injections of bupivacaine HCl in 0.9% saline (1.5 mg/ml) in those specific periarticular locations. Paw withdrawal threshold indicate similar pain relief initially followed by prolonged pain relief in the PEA nanoparticles+bupivacaine treated group as compared with the periarticular bupivacaine alone group as depicted in FIG. 10 demonstrating the unique advantages of the combination approach. FIG. 10 depicts electronic Von Frey measurements done with treatment groups using bupivacaine HCL periarticularly injected at 2 locations alone and periarticular injection of PEA nanoparticles with bupivacaine HCL at 2 locations. The combination approach showed similar pain relief compared to bupivacaine alone group initially followed by prolonged pain relief demonstrating the unique advantages of the combination approach.

### Example 9

This example was conducted to determine the effect of increasing the dosage of the local anesthetic as commonly 5 mg/mL bupivacaine is used clinically. Increasing the dose of bupivacaine from 1.5mg/ml to 3 mg/mL significantly reduced the efficacy of local anesthetics presumably due to the associated toxic side effects. However, adding PEA nanoparticles to the bupivacaine formulation mitigated the adverse effects and synergistically enhanced the analgesic capabilities of the local anesthetic. For example, female Sprague Dawley Rats were injected with 30 μL of mono-iodoacetate solution (MIA) to induce OA.

After 24 hours, one group was injected with bupivacaine (3 mg/ml, 50 μL/location) at one location: superior lateral. The other group of rats were injected with 50 L of PEA nanoparticles+bupivacaine HCl (3 mg/ml) in 0.9% saline at superior lateral location and 100 μL of PEA nanoparticles in 0.9% saline in inferior medial location.

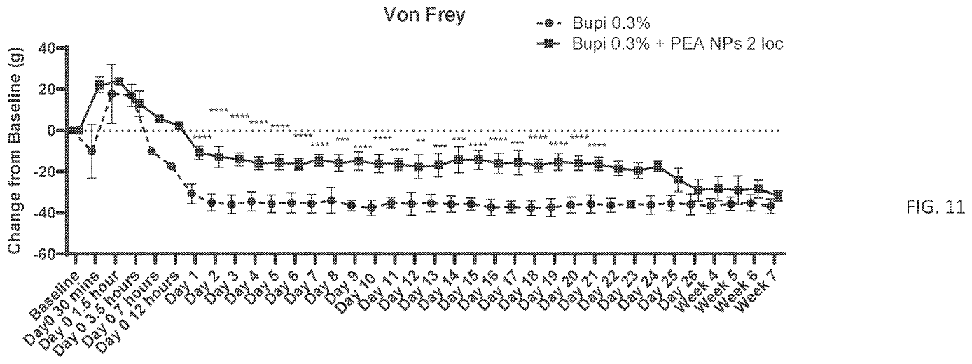

Pain response was measured using Von Frey measurements. Von Frey measurements indicate significantly higher pain relief in the PEA nanoparticles and high dose of bupivacaine HCL group compared to high dose of bupivacaine HCL alone. The PEA nanoparticles with high dose of bupivacaine HCL extended pain relief more than 4 weeks compared to a few hours in the high dose bupivacaine HCL alone group. FIG. 11 depicts electronic Von Frey measurements done with treatment groups using high dose of bupivacaine HCL alone at one location (N=4) and periarticular PEA nanoparticles at two locations with high dose bupivacaine HCL at 1 location (N=5). It shows the significantly enhanced hypoalgesic and analgesic effect of the combination approach and thereby demonstrates the enhanced therapeutic efficacy of the combination approach. In FIG. 11, ‘**’ indicates p<0.01, while ‘***’ indicates p<0.001 and ‘*****’ indicates that p<0.0001 in a two-tailed t-test.

From the in vitro data above it may be seen that palmitoylethanolamide nanoparticles display a significantly higher anti-inflammatory effect compared to completely soluble palmitoylethanolamide thus demonstrating the unique efficacy of the nanoparticle formulation. The palmitoylethanolamide nanoparticles also reduced inflammation in-vitro in a dose dependent manner. In addition, the injection of palmitoylethanolamide nanoparticles locally in-vivo significantly reduced local knee inflammation. Unique synergistic effects in reducing inflammation and enhancing the analgesic effect of a local anesthetic were observed when palmitoylethanolamide nanoparticles were combined with a local anesthetic in (bupivacaine HCl) in-vivo studies.

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

Compounds and materials are described using standard nomenclature. Unless defined otherwise, all technical and scientific terms used herein have the same meaning as is commonly understood by one of skill in the art to which this invention belongs. The following terms are used to describe the invention of the present disclosure. In instances where a term is not specifically defined herein, that term is given an art-recognized meaning by those of ordinary skill applying that term in context to its use in describing the present disclosure.

The use of the terms “a” and “an” and “the” and similar referents (especially in the context of the following claims) are to be construed to cover both the singular and the plural, unless otherwise indicated herein or clearly contradicted by context. By way of example, “an element” means one element or more than one element.

As used herein, the term “substantially” means to a great or significant extent, but not completely.

It should also be understood that, in certain methods described herein that include more than one step or act, the order of the steps or acts of the method is not necessarily limited to the order in which the steps or acts of the method are recited unless the context indicates otherwise. Furthermore, the terms first, second, etc., as used herein are not meant to denote any particular ordering, but simply for convenience to denote a plurality of, for example, layers.

The terms “comprising”, “having”, “including”, and “containing” are to be construed as open-ended terms (i.e., meaning “including, but not limited to”) unless

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a nonlimiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “one or more,” as used herein, means at least one, and thus includes individual components as well as mixtures/combinations of the listed components in any combination.

The terms “about” or “approximately,” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Other than in the operating examples, or where otherwise indicated, all numbers expressing quantities of ingredients and/or reaction conditions are to be understood as being modified in all instances by the term “about,” meaning within 10% of the indicated number (e.g., “about 10%” means 9%-11% and “about 2%” means 1.8%-2.2%).

Recitation of ranges of values are merely intended to serve as a shorthand method of referring individually to each separate value falling within the range, unless otherwise indicated herein, and each separate value is incorporated into the specification as if it were individually recited herein. All ranges disclosed herein include both end points as discrete values as well as all integers and fractions specified within the range. For example, a range of 0.1-2.0 includes 0.1, 0.2, 0.3, 0.4 . . . 2.0. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed.

No language in the specification should be construed as indicating any non-claimed element as essential to the practice of the invention as used herein. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,”1% to 5%,“2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

As used herein, the term “administering” means the actual physical introduction of a composition into or onto (as appropriate) a subject, a host, or cell. Any and all methods of introducing the composition into the subject, host or cell are contemplated according to the invention; the method is not dependent on any particular means of introduction and is not to be so construed. Means of introduction are well-known to those skilled in the art, and also are exemplified herein. “Providing” means giving, administering, selling, distributing, transferring (for profit or not), manufacturing, compounding, or dispensing.

The term “subject” or “patient” is used herein to refer to an animal, such as a mammal, including a primate (such as a human, a non-human primate, e.g., a monkey, and a chimpanzee), a non-primate (such as a cow, a pig, a camel, a llama, a horse, a goat, a rabbit, a sheep, a hamster, a guinea pig, a cat, a dog, a rat, a mouse, and a whale), a bird (e.g., a duck or a goose), and a shark. In an embodiment, the subject or patient is a human subject or a human patient, such as a human being treated or assessed for a disease, disorder or condition, a human at risk for a disease, disorder or condition, a human having a disease, disorder or condition, and/or human being treated for a disease, disorder or condition as described herein. In one embodiment, the subject is about 1, 2, 3, 4, 5, 6, 7, 8, 9, or 10 years of age. In another embodiment, the subject is about 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100 years of age.

Values and ranges intermediate to the above recited ranges are also intended to be part of this invention. In addition, ranges of values using a combination of any of the above-recited values as upper and/or lower limits are intended to be included. As used herein, a subject is “in need of treatment” if such subject would benefit biologically, medically, or in quality of life from such treatment. A subject in need of treatment does not necessarily present symptoms, particular in the case of preventative or prophylaxis treatments.

While the invention has been described with reference to some embodiments, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiments disclosed as the best mode contemplated for carrying out this invention, but that the invention will include all embodiments falling within the scope of the appended claims.

1. An analgesic composition comprising:
a therapeutically effective amount of at least one endocannabinoid, at least one cannabinoid, or a combination of the endocannabinoid and cannabinoid; where the endocannabinoid comprises one or more endocannabinoids, one or more analogues of the endocannabinoids, one or more pharmaceutically acceptable salts of the endocannabinoid, or a combination thereof; where the cannabinoid comprises one or more cannabinoids, one or more analogues of the cannabinoids, one or more pharmaceutically acceptable salts of the cannabinoid, or a combination thereof; where at least one of the endocannabinoid or the cannabinoid is in the form of particles.

2. The analgesic composition of claim 1, further comprising a local anesthetic.

3. The analgesic composition of claims 1, where the analgesic composition is an aqueous suspension of particles and where the particles are nanoparticles and/or microparticles.

4. The analgesic composition of claim 1, where the local anesthetic is in the form of particles.

5. The analgesic composition of claim 1, where the endocannabinoid is present in the analgesic composition in an amount of about 1 to about 99 wt %, based on a total weight of the analgesic composition.

6. The analgesic composition of claim 2, where the endocannabinoid reduces toxic effects of the local anesthetic.

7. The analgesic composition of claim 2, where the endocannabinoid increases and prolongs an analgesic efficacy of local anesthetic.

8. The analgesic composition of claim 1, wherein a combination of the endocannabinoid and/or cannabinoid and the local anesthetic reduces inflammation faster than a therapeutically effective amount of the local anesthetic.

9. The analgesic composition of claim 1, wherein the nanoparticles have a particle size of 0.5 to 100 nanometers and where the microparticles have a particle size of about 100.1 to about 100,000 nanometers.

10. The analgesic composition of claim 1, wherein the endocannabinoid is palmitoylethanolamide.

11. The analgesic composition of claim 1, further comprising a lipid or a phospholipid.

12. The analgesic composition of claim 1, wherein the lipid is 1,2-dipalmitoyl-sn-glycero-3-phosphocholine.

13. The analgesic composition of claim 1, wherein the aqueous solution comprises saline.

14. A method of manufacturing endocannabinoid and/or cannabinoid particles, the method comprising:
dissolving the endocannabinoid and/or cannabinoid and a lipid in a first solvent;
evaporating the first solvent to leave behind a powder or film;
adding to the powder or film an aqueous solution;
heating the aqueous solution containing the powder and the film to a temperature greater than about 100° C. or to a temperature higher than the melting point of the lipid; and
agitating the aqueous solution with a rotary agitator at about 5,000 to about 15,000 revolutions per minute to form the endocannabinoid and/or cannabinoid particles; where the particles are nanoparticles and/or microparticles.

15. The method of claim 14, where the first solvent is an organic solvent, and wherein the aqueous solution comprises saline.

16. A method of reducing joint inflammation, swelling and/or pain in a subject, the method comprising:
administering a therapeutically effective amount of at least one endocannabinoid, at least one cannabinoid, or a combination of the endocannabinoid and cannabinoid to the subject; where at least one of the endocannabinoid or the cannabinoid is in the form of a nanoparticle and/or microparticle; and wherein the endocannabinoid comprises one or more endocannabinoids, one or more analogues of the endocannabinoids, one or more pharmaceutically acceptable salts of the endocannabinoid, or a combination thereof; where the cannabinoid comprises one or more cannabinoids, one or more analogues of the cannabinoids, one or more pharmaceutically acceptable salts of the cannabinoid, or a combination thereof.

17. The method of claim 16, wherein the therapeutically effective amount is administered in a single dose or multiple doses periarticularly or intraarticularly or locally around the site of pain/injury.

18. The method of any one of claim 17, wherein the single dose reduces pain by an amount equivalent to two or more doses administered periarticularly.

19. The method of claim 16, further comprising a local anesthetic.

20. The method of claim 16, wherein the endocannabinoid is palmitoylethanolamide.
